(19) United States
(12) Patent Application Publication
(10) Pub. No.: US 2016/0357937 A1
(43) Pub. Date: Dec. 8, 2016

(54) MOBILE APPLICATION FOR MAIL ORDER PHARMACY PROGRAM

(71) Applicant: Humana Inc., Louisville, KY (US)

(72) Inventors: Nick Walter, Louisville, KY (US); Andrew Foster, Coxs Creek, KY (US); Dustin Hamilton, Brookfield, IL (US); Ryan Reed, Louisville, KY (US)

(21) Appl. No.: 14/517,129

(22) Filed: Oct. 17, 2014

(51) Int. Cl. G06F 19/00; G06Q 10/08; G06Q 30/06

(52) U.S. Cl. CPC G06F 19/3456; G06Q 30/0635; G06Q 10/083

(57) ABSTRACT

A mobile application for a mail order pharmacy program is disclosed. After enrolling in a mail order pharmacy program and receiving one or more prescriptions through the mail, members may use the mobile application to order prescription refills. Members request a prescription refill by scanning a barcode on a prescription vial for a prior fill. In an example embodiment, the barcode comprises an order number and an inventory number from a prior fill. The barcode comprises data that is used in processing the prescription through an automated mail order pharmacy system and that further facilitates reordering using the mobile application. Once received at the mail order pharmacy, the order number and product number from the barcode are used to locate the previous order for the prescription and to issue another order for the prescription that is processed on the automated dispensing line of the mail order pharmacy system.

### CROSS-REFERENCE TO RELATED APPLICATIONS

None.

### BACKGROUND

Many health benefit plan providers offer their plan members the option of obtaining their prescription drugs by mail. For members with chronic conditions or other conditions that require maintenance drugs, a mail order prescription program is an attractive benefit because it is more convenient for members and typically less expensive than obtaining prescription drugs at a neighborhood pharmacy. For many drugs, members have the option of purchasing a drug fill in a 60-day or even a 90-day supply at a lower cost than a 30-day supply.

Many health benefit plan providers offer their members a web-based “self-serve” option for enrolling in a mail order pharmacy program and refilling prescriptions through the mail order pharmacy program. Members access a web portal that provides functionality for submitting refill requests for prescriptions that are filled through the mail order pharmacy. The member's eligible prescriptions appear in a list and the member is provided with a refill option for each prescription. After selecting the prescriptions to be refilled, an ordering application at the server acknowledges the member's request or requests and provides confirmation that the refill request has been received. The member may receive periodic updates (e.g., a shipping notification, a tracking number, etc.) as the request is processed and the prescription is filled and shipped from the mail order pharmacy.

Although web-based prescription ordering is a convenient way for a member of a health plan to obtain refills, it typically requires access to a personal computer as well as an Internet connection. The ability to place an order is limited to those times when the member has access to a computer and Internet connection such as when at home or at work. Smart phones, tablets, and other portable devices can be used at almost any time and anywhere to access the mail order pharmacy portal through a browser but because the devices have small screens, navigating a web site can be cumbersome. To address this problem, many prescription retailers offer prescription ordering mobile applications that provide features and functionality for entering and viewing information about prescriptions and placing orders for prescriptions filled through a local retail pharmacy. As a result, the applications employ a “scan to pick up” function to facilitate processing of the prescription fill by a retail pharmacy that a user then visits to obtain the refill. The process of dispensing prescriptions by mail is different and therefore, there is a need for mobile application to facilitate “scan to mail” processing of prescription refills.

### SUMMARY

The present disclosure is directed to a mobile application for a mail order pharmacy program. After enrolling in a mail order pharmacy program and receiving one or more prescriptions through the mail, members may use the mobile application to order prescription refills. The disclosed mobile application allows a member to take advantage of the convenience and cost savings of a mail order pharmacy program. The disclosed mobile application further enhances the convenience of the mail order pharmacy program by increasing the convenience of ordering refills. Members can place orders at their convenience and receive confirmations, shipping and tracking information, and other information through the mobile application.

In an example embodiment, members request a prescription refill by scanning a barcode on a prescription bottle for a prior fill. In an example embodiment, the barcode comprises an order number and an inventory number from a prior fill. The barcode comprises data that is used in processing the prescription through an automated mail order pharmacy system and that further facilitates reordering using the mobile application. Once received at the mail order pharmacy, the order number and inventory number from the barcode are used to locate the previous order for the prescription and to issue another order for the prescription that is processed on the automated dispensing line of the mail order pharmacy system.

### BRIEF DESCRIPTION OF THE DRAWINGS

FIG. 1 is an automated dispensing line schematic diagram for a mail order pharmacy according to an example embodiment.

FIG. 2 is a flow diagram for a mobile application order process according to an example embodiment.

FIG. 3 is a flow diagram for a mobile application available refills process according to an example embodiment.

FIG. 4 is a flow diagram for a mobile application refills selection process according to an example embodiment.

FIG. 5 is a sample order screen for a mobile application according to an example embodiment.

FIG. 6 is a sample “Scan to Refill” screen for a mobile application according to an example embodiment.

FIG. 7 is a sample “Successful Scan” screen for a mobile application according to an example embodiment.

FIG. 8 is a sample “Manual Entry” screen for a mobile application according to an example embodiment.

FIG. 9 is a sample “Invalid Prescription Number” screen for a mobile application according to an example embodiment.

### DETAILED DESCRIPTION

In an example embodiment, the mail order pharmacy uses automated systems and dispensing lines to process and ship a high volume of prescriptions on a daily basis. Each prescription medication is dispensed into a vial or other container or bottle labeled with barcoded data from an electronic order that comprises the order number, patient identifying and shipping data, and for each item in the order, a drug identifier (e.g., by NDC), a dosage, a quantity and an inventory number. Each inventory number identifies the specific drug and dosage. Each medication is dispensed in its own vial and in many instances, multiple vials are combined into a single package and shipped to a single address for a member with one or more chronic conditions requiring multiple medications. The automated dispensing system determines which vials should be combined into a single package for shipping to a single address and routes them accordingly.

In an example embodiment, electronic order data for a prescription comprises the following information.

TABLE 1
Electronic Prescription Order Data

Mail Order Pharmacy Order Number
Mail Order Pharmacy Account Number
Patient Information

Name
Member Date of Birth
Member Identifier

Physician Information

Name
Address
City/State/Zip
Telephone Number
Fax Number

Member Shipping Information

Name
Address
City/State/Zip
Telephone Number

Drug Name (NDC), Dosage, Quantity,
Inventory Number for each prescribed
medication

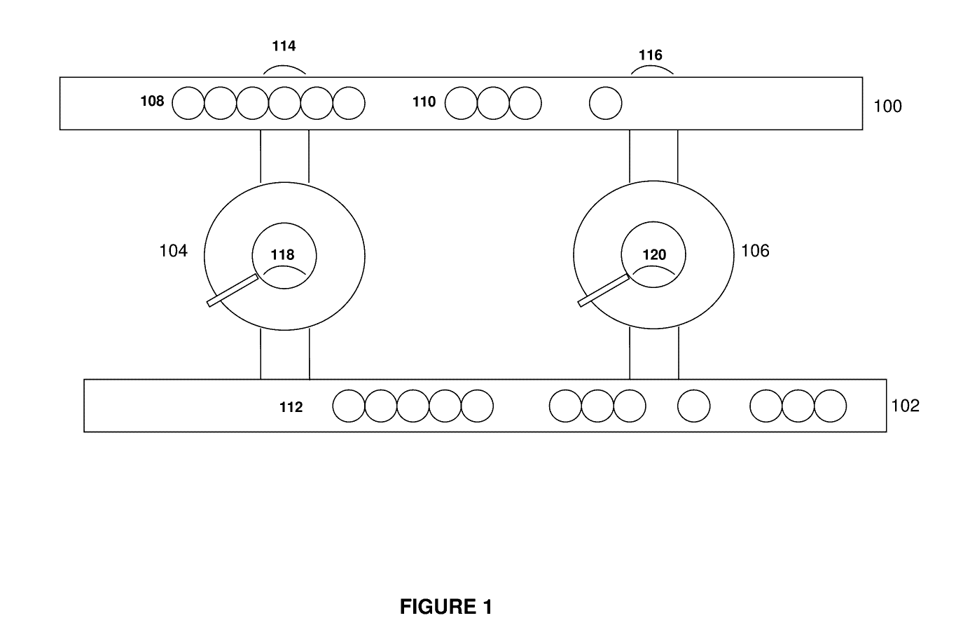

Referring to FIG. 1, an automated dispensing line schematic diagram for a mail order pharmacy according to an example embodiment is shown. The line may comprise a plurality of conveyors 100 102 for transporting vials 108 110 112 to automated tablet counters (ATC) 114 116, sorting stations 104 106, and other locations on the line. In an example embodiment, each vial is transported in a puck that contains the vial and that comprises an RFID tag for use in routing the vial on the line and dispensing a medication from an ATC into the vial. A plurality of stations (ATCs, diverters, etc.) on the line comprise an RFID reader that reads the identifying information for a puck/vial and transmits it to a line control application that facilitates pharmaceutical order processing. The line control application receives order data (e.g., drug identifying data such as a National Drug Code (NDC), dosage, and quantity) as well as an RFID identifier for a puck that contains a vial in which the drug will be dispensed. Pucks are then routed by the line control application to appropriate stations (e.g., ATC with identified drug, sorting station, packaging station) so the drugs can be dispensed into the vials and shipped to members. Vials are routed using the conveyors 100 102 and are diverted to various stations on the line or other conveyors using mechanical diverting devices 114 116 118 120 that “push” a vial from one direction of travel to another.

Each vial also receives a barcode label with order details encoded in the barcode. In an example embodiment, the barcode comprises an order number and an inventory number (e.g., product or SKU number) for a drug. The vial barcodes are scanned during packaging to ensure the vial is placed in a package with the correct shipping information. Order details are also barcoded on a shipping insert for the package. In an example embodiment, the shipping insert barcode is scanned by a packaging operator and the vial barcodes are scanned automatically as vials are routed on the automated dispensing line and automatically dropped into a mailer. The shipping insert and vial barcode data is compared to ensure the data on the barcodes matches the electronic order data. Once the match is confirmed, the shipping insert is added to the mailer with the vials, and the mailer is sealed and processed for shipping to the member.

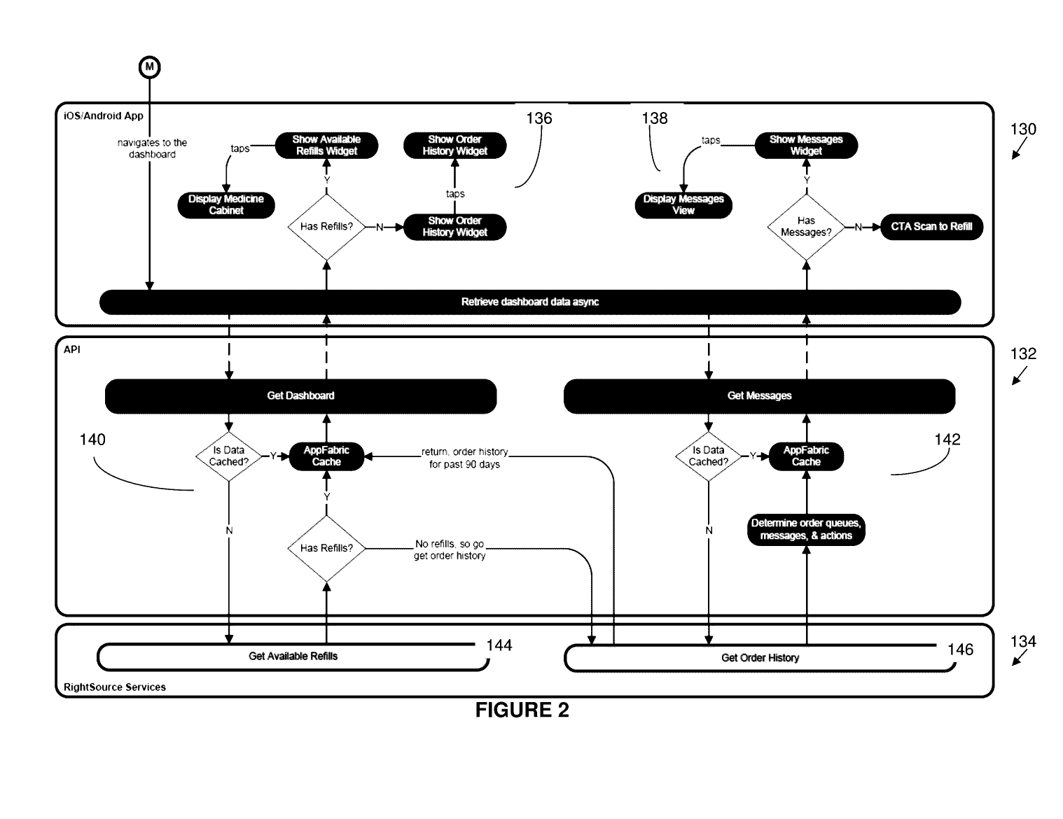

FIG. 2 is a flow diagram for a mobile application refills process according to an example embodiment. The refills process comprises mobile application 130, API 132, and server-side 134 modules for generating a display comprising a list of available refills and order status messages. When the member navigates to a “dashboard” screen, the member's available refills 144 and order history 146 are retrieved from a mail order pharmacy server. The member's available refills 140 and order messages 142 are organized for display in the mobile application screen 136 138.

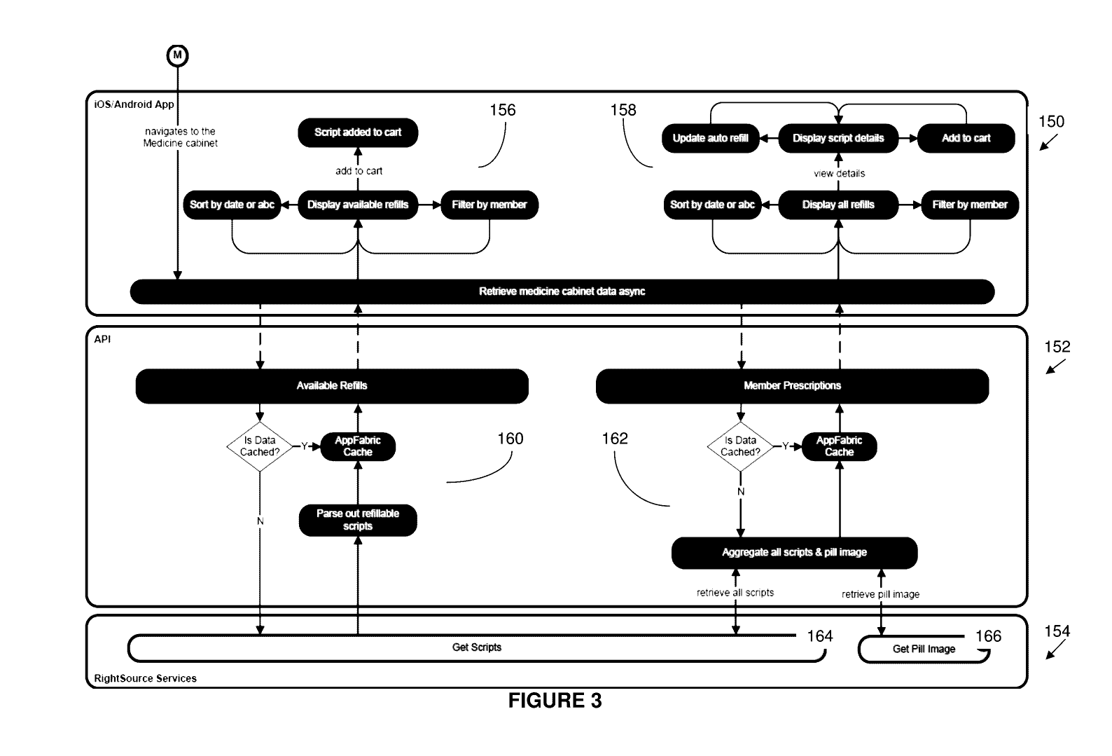

FIG. 3 is a flow diagram for a mobile application order process according to an example embodiment. The order process comprises mobile application 150, API 152, and server-side 154 modules for generating a display comprising prescription details. Prescription data 164 and pill image data 166 is retrieved from a server at the mail order pharmacy. Data for the member's available refills is organized for display 160. Additionally, details for all of the member's prescriptions are organized for display 162. Available refills 156 are displayed by the mobile application as well as all refills 158 which may include refills for dependents covered under the member's health plan.

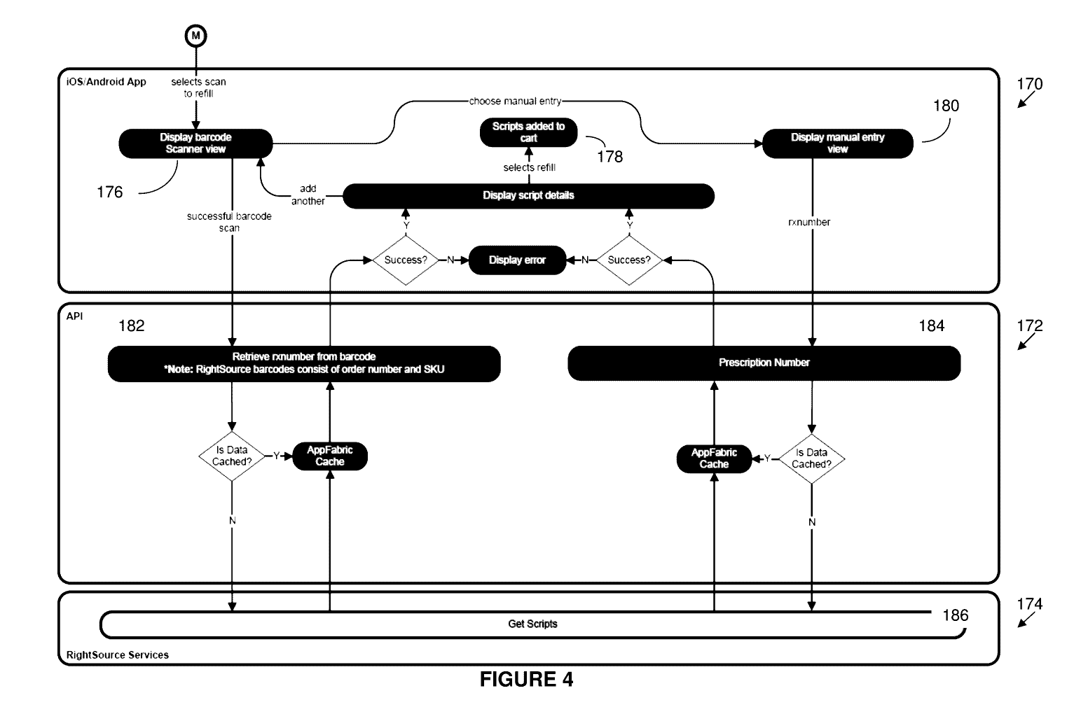

FIG. 4 is a flow diagram for a mobile application order fulfillment process according to an example embodiment. The order fulfillment process comprises mobile application 170, API 172, and server-side 174 modules for initiating fulfillment of a prescription refill. After selecting a “scan to refill” option 176, the member scans the barcode of a prescription to be refilled. The prescription number is retrieved from the barcode scan 182. In an example embodiment, the prescription number comprises an order number and an inventory (e.g., product or SKU) number. At the mail order pharmacy, the order number is linked to the member's data including applicable health plan, covered dependents, and shipping address. The inventory number identifies the specific item to be refilled. The data for the prescription number is located in a cache or retrieved from the server 186. The prescription details are then displayed by the mobile application 178. The member selects a “refill” option to add the prescription to a cart 178. Alternatively, the member has the option of entering the prescription number manually 180. After selecting the manual option, the member enters the prescription number 184. The data for the prescription number is located in a cache or retrieved from the server 186. The prescription details are then displayed by the mobile application 178. The member selects a “refill” option to add the prescription to a cart 178.

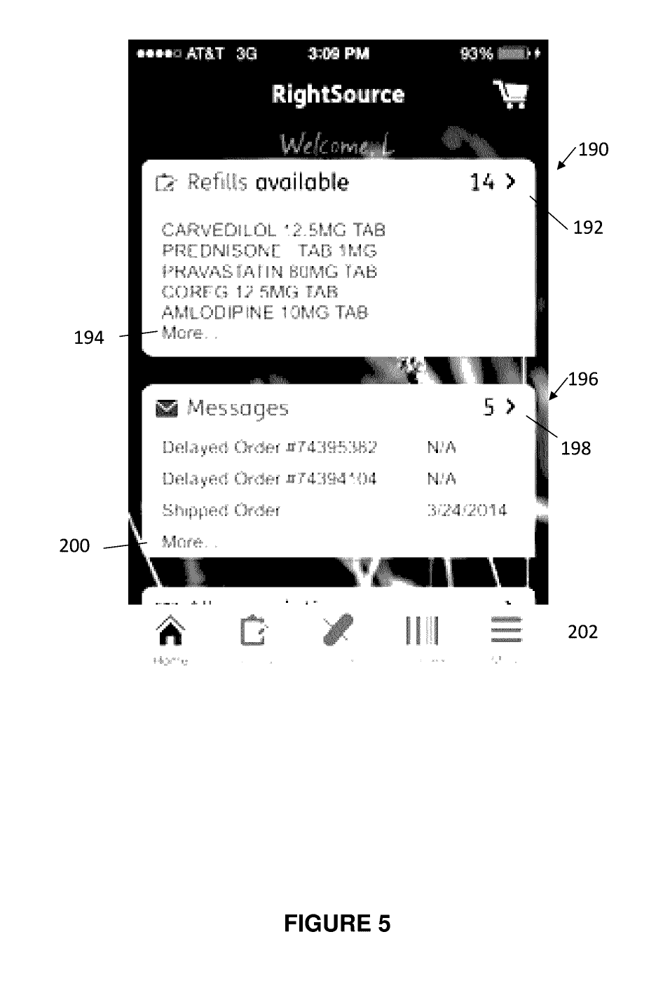

After the member receives a first fill of the prescription, the mobile application may be used to order subsequent refills. Referring to FIG. 5, a sample order screen for a mobile application according to an example embodiment is shown. In one portion of the screen, the member's prescriptions that are available for refill are displayed 190. The total number of available prescriptions appears in the upper right corner 192 and the member may view additional prescriptions by selecting the “More” option 194. In another portion of the screen, status messages for orders are displayed 196. The total number of messages appears in the upper right corner 198 and the member may view additional messages by selecting the “More” option 200. Another portion of the screen comprises additional options for navigating to other screens of the mobile application 202.

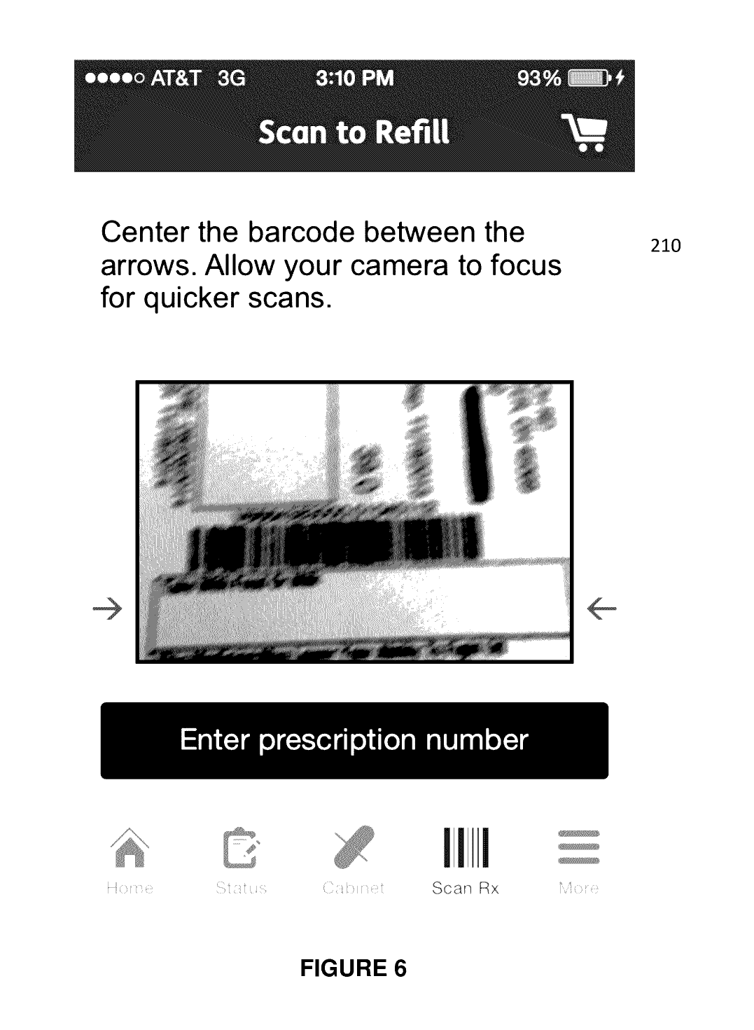

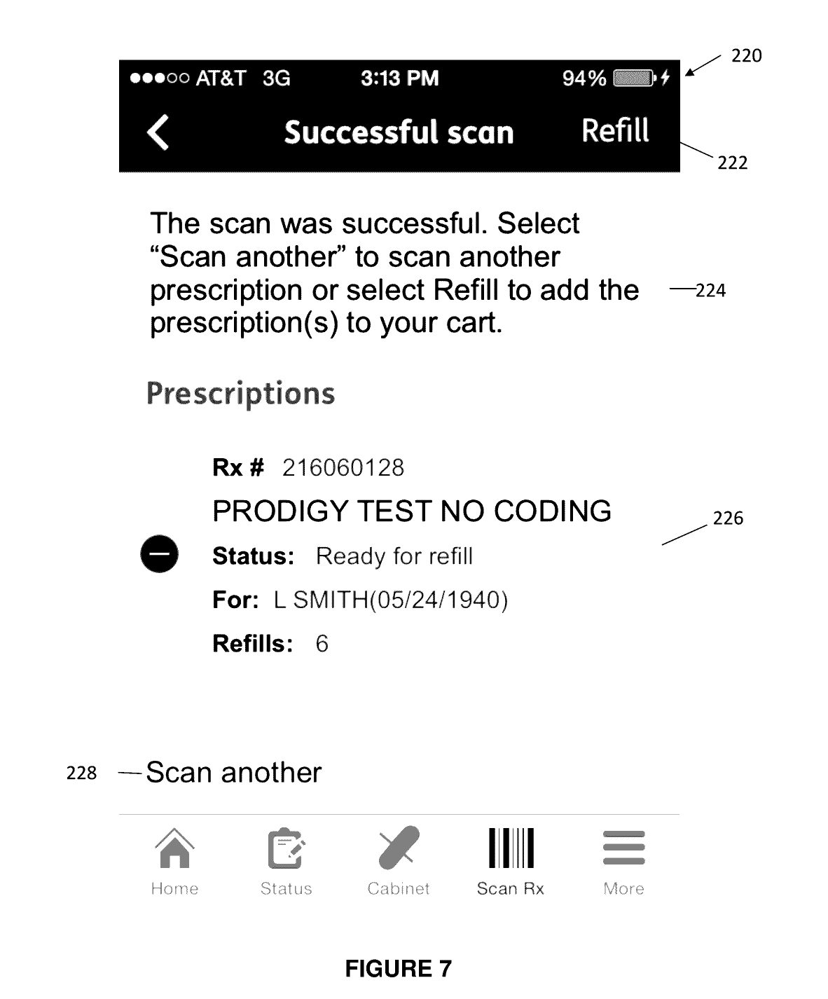

After selecting a prescription to refill, a “Scan to Refill” screen as shown in FIG. 6 appears. The screen comprises an instruction to the member to focus the camera on the vial barcode 210. If the scan is successful, a “Successful Scan” screen as shown in FIG. 7 appears. The screen 220 comprises several sections. In one section 226, the prescription number, a status message, patient data, and the remaining number of refills are displayed. The screen further comprises an instruction section 224 with a message to scan another prescription by selecting a “Scan another” option 228 or to add the prescription to a cart by selecting a “Refill” option 222.

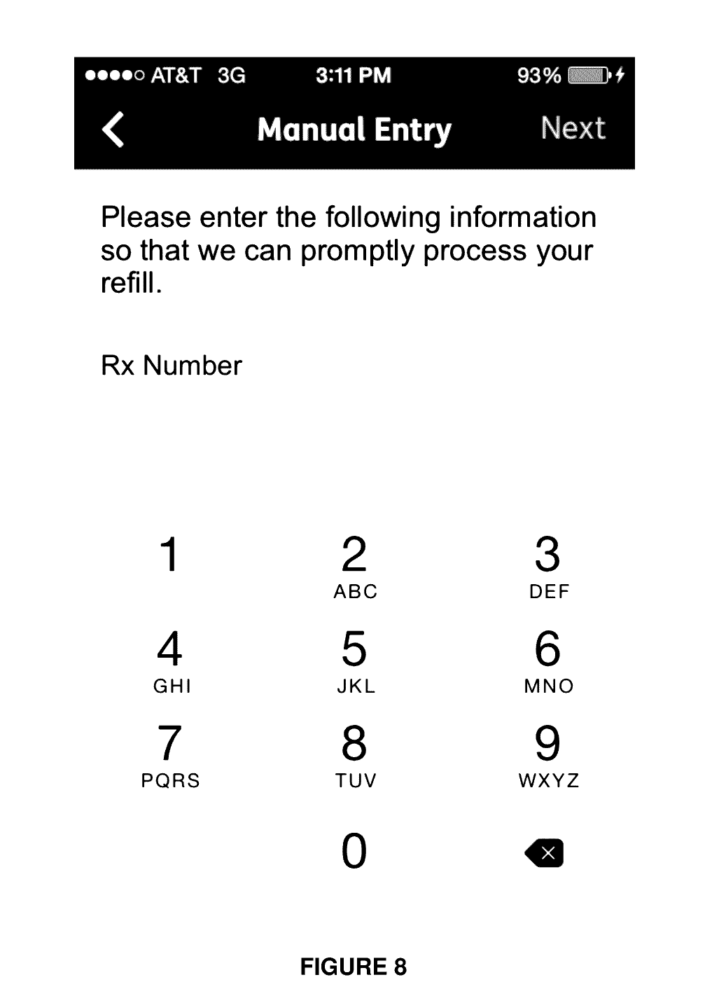

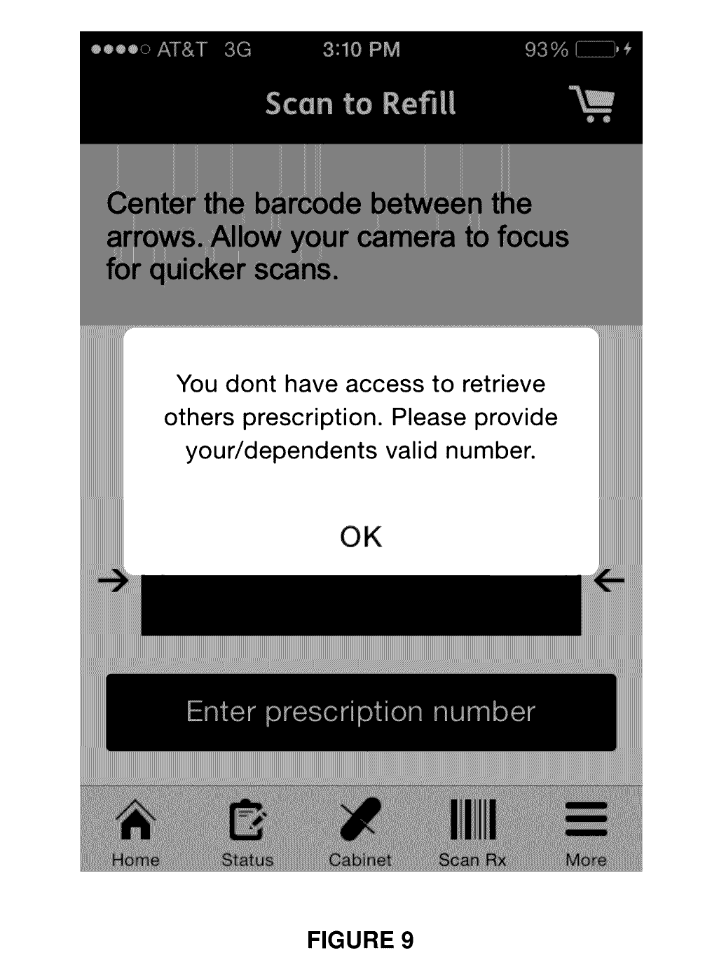

If the scan is not successful, the member has the option of manually entering a prescription number on a “Manual Entry” screen as shown in FIG. 8. A keypad is displayed and the member enters the prescription number from the vial. If an invalid prescription number is entered using the scan option or manual entry option, an error message is displayed to the member as shown in FIG. 9.

Once the refill order details are received by the mobile application and payment processing is completed, a refill request for each selected prescription is transmitted by the mobile application to the mail order pharmacy prescription ordering system. The order number from the refill order data is used to retrieve the member's previous order from the prescription ordering system database to determine the order details for processing of the order on the automated dispensing line using the line control application. The inventory, product, or SKU number from the refill order data identifies the specific prescription drug to be refilled. A vial for the prescription refill is added to a puck and routed using the puck RFID tag. The drug is dispensed from an ATC into the vial and the vial is routed by the line control application through dispensing and packaging for shipment to the member. Additional refill orders may be initiated through the mobile application for processing at the mail order pharmacy.

While certain embodiments of the disclosed mobile application for a mail order pharmacy are described in detail above, the scope of the invention is not to be considered limited by such disclosure, and modifications are possible without departing from the spirit of the invention as evidenced by the claims. For example, elements of the user interface and screen layouts may be varied and fall within the scope of the claimed invention. Various aspects of user interactions and presentation of data may be varied and fall within the scope of the claimed invention. One skilled in the art would recognize that such modifications are possible without departing from the scope of the claimed invention.

What is claimed is:

1. A computerized method for ordering a prescribed drug comprising computer instructions to:
(a) receive a prescription order comprising:
(i) member identifying information for a member of a health benefits plan;
(ii) member shipping information for shipping mail order prescriptions to said member;
(iii) an order number; and
(iv) an inventory number for a drug to be dispensed to said member;
(b) initiate fulfillment of said prescription order on an automated dispensing line by:
(i) adding to a first vial comprising said drug a first barcode comprising:
(1) said order number; and
(2) said inventory number;
(ii) adding said first vial to a first shipping mailer comprising said member identifying information and said member shipping information;
(c) receive from a mobile application a refill request for said prescription comprising said barcode;
(d) locate said order number and said inventory number from said barcode;
(e) initiate fulfillment of said refill request for said drug on said automated dispensing line by:
(i) adding to a second vial comprising said drug a second barcode comprising said order number and said inventory number; and
(ii) adding said second vial to a second shipping mailer comprising said member identifying information and said member shipping information.

2. The computerized method of claim 1 wherein said refill request is selected from a list of available refills.

3. The computerized method of claim 1 further comprising transmitting for display by said mobile application an order status message for said refill request.

4. The computerized method of claim 1 further comprising receiving from said mobile application a second refill request for said drug.

5. The computerized method of claim 4 wherein said second refill request for said drug is initiated from a manual entry of a prescription number.

6. The computerized method of claim 1 wherein said refill request is for a covered dependent under said health benefits plan.

7. The computerized method of claim 1 wherein said refill request is one of a plurality of refill requests in a shopping cart for said mobile application.

8. A computerized system for ordering a prescribed drug comprising:
(A) a server executing computer instructions to:
(1) receive a prescription order comprising:
(a) member identifying information for a member of a health benefits plan;
(b) member shipping information for shipping mail order prescriptions to said member;
(c) an order number; and
(d) an inventory number for a drug to be dispensed to said member;
(2) initiate fulfillment of said prescription order on an automated dispensing line by:
(a) adding to a first vial comprising said drug a first barcode comprising:
(i) said order number; and
(ii) said inventory number;
(b) adding said first vial to a first shipping mailer comprising said member identifying information and said member shipping information;
(3) process refill requests for said drug comprising said barcode by;
(a) locating said order number and said inventory number from said barcode;
(b) initiating fulfillment of said refill request for said drug on said automated dispensing line by:
(i) adding to a second vial comprising said drug a second barcode comprising said order number and said inventory number; and
(ii) adding said second vial to a second shipping mailer comprising said member identifying information and said member shipping information; and
(B) a mobile phone executing a mobile application for transmitting to said server a refill request comprising said barcode.

9. The computerized system of claim 8 wherein said refill request is selected from a list of available refills.

10. The computerized system of claim 8 wherein said mobile application displays an order status message for said refill request.

11. The computerized system of claim 8 wherein said mobile application further transmits a second refill request for said drug.

12. The computerized system of claim 11 wherein said second refill request for said drug is initiated from a manual entry of a prescription number.

13. The computerized system of claim 8 wherein said refill request is for a covered dependent under said health benefits plan.

14. The computerized system of claim 8 wherein said refill request is one of a plurality of refill requests in a shopping cart for said mobile application.

15. An apparatus for ordering a prescribed drug comprising a mobile phone application including instructions executed by a processor to:
(a) receive from a user a selection of an available refill for a prescription drug;
(b) transmit to a mail order pharmacy server a request to fill said available refill for said prescription drug, said request comprising a barcode number identifying:
(i) an order number for said prescription drug; and
(ii) an inventory number for said prescription drug;
(c) receive from said mail order pharmacy server an order status message for said request to fill said available refill for said prescription drug; and
(d) display for said user said order status message.

16. The apparatus of claim 15 wherein said order number for said prescription drug transmitted to said mail order pharmacy server is used at said mail order pharmacy server to locate:
(a) member identifying information for a member of a health benefits plan;
(b) member shipping information for shipping mail order prescriptions to said member; and
(c) drug identifying information for at least one prescription to be dispensed to said member.

17. The apparatus of claim 15 wherein said selection of an available refill for a prescription drug is made from a list of available refills.

18. The apparatus of claim 15 wherein said order status message is selected from the group consisting of delayed order status messages and shipped order status messages.

19. The apparatus of claim 15 further comprising instructions executed by said processor to prompt said user for manual entry of a prescription number for a prescription drug to be refilled.

20. The apparatus of claim 19 further comprising instructions executed by said processor to transmit said prescription number to said mail order pharmacy server.
